(19) United States
(12) Patent Application Publication
(10) Pub. No.: US 2016/0325470 A1
(43) Pub. Date: Nov. 10, 2016

(54) DEVICE AND METHOD FOR PRODUCING OBJECTS FROM BIODEGRADABLE MATERIALS

(71) Applicant: Matthijs BRUGMANS, Rossendaal (NL)

(72) Inventor: Matthijs BRUGMANS, Roosendaal (NL)

(21) Appl. No.: 15/108,421

(22) PCT Filed: Mar. 10, 2015
(86) PCT No.: PCT/IB2015/000306

§ 371 Date: Jun. 27, 2016

(30) Foreign Application Priority Data
Mar. 13, 2014 (BE) 2014/0167

(51) Int. Cl. B29C 39/00; G01N 33/00; B29C 39/36; B29C 33/28; B29C 39/44

(52) U.S. Cl. CPC B29C 39/003; B29C 33/28; B29C 39/44; B29C 39/36; G01N 33/0098; B29K 2003/00; B29K 2995/006; B29L 2031/712

(57) ABSTRACT

Device (1) for producing objects from biodegradable materials consisting of a storage tank (5) with solid or liquid biodegradable material that is connected to a distribution line (6) along which a series of baking forms (7) with an individualizable form are filled, and whereby each baking form (7) goes through an individualizable computer-controlled process program that determines the filling time, pressure and temperature, and afterwards automatically opens the baking form (7) and removes the object formed, after which the next piece can be pressed or cast in the same baking form (7).

The present invention relates to a device for producing objects from biodegradable materials and a method whereby such a device is used.

More specifically, the invention is intended for producing objects from agricultural products such as potato starch, maize, cassava, rice husks and similar that are biodegradable.

It is known that biodegradable products are recyclable by nature and can replace synthetic plastics that give rise to an ever greater waste mountain of toxic and non-biodegradable products, generally originating from plastic packaging.

The plastic waste problem affects all countries in the world, but in the rapidly economically developing countries in particular the environment is threatened by this problem, such that the waste problem has become the subject of many international conferences.

For example it is possible to produce utensils such as noodle cups or lunchboxes from biodegradable materials. The daily consumption of each of these objects in China alone is approximately 200 million units per day.

A great advantage of biodegradable materials, such as starch for example, is that the objects made from them can be recycled after use, either as cheap natural energy or as fertiliser in agriculture. These biodegradable materials are 100% degradable and dissolve in the soil within 20 days.

Starch is a suitable material for producing food packaging such as hamburger boxes, cheese or meat dishes, fruit baskets and other carriers for dry foodstuffs.

Rice husks are primarily used for products with a high water content and for food dishes such as those used for aircraft meals.

In addition to food packaging, biodegradable materials are not only suitable for packaging smaller volumes, but also for higher value products such as electronic consumer articles.

Biodegradable materials have been developed in the meantime to a point where mass production for the consumer market is possible.

Traditionally solid products such as starch are processed into a paste from which objects can be pressed in a baking form.

But more liquid products such as defragmented lactic acid or citric acid mixed with glycerol, enzymes and other vegetable materials can be processed for the production of objects. In this case the liquid is pumped under a controlled pressure by which the baking form is filled.

Objects are often produced from synthetic polymers by extruding molten polymer in the desired form or baking form, after which the object formed is taken out of the baking form after cooling.

This extrusion technique cannot be used for biodegradable polymers because they are not suitable for melting and thus first have to be pressed or poured into a baking form, before heating or baking them in the baking form in order to give them their final shape.

A problem occurs when large quantities of objects must be produced from a biodegradable material. The use of a single baking form, as is usual in extrusion technology, limits the speed at which a large number of objects, for example packaging, can be produced.

An additional problem occurs when series of different objects such as different forms of packaging must be produced and the baking form must be repeatedly replaced by a different one. The machine thereby has to be stopped and converted, which for small series of a certain packaging in particular detrimentally affects the cost per packaging and impedes the automatability of the production.

The purpose of the present invention is to provide a solution to the aforementioned and other disadvantages by providing a device for producing objects from biodegradable materials that enables a number of objects of the same or a different form to be produced simultaneously from solid or liquid biodegradable materials by filling and heating baking forms whereby each baking form not only has an individualisable form, but also goes through a computer-controlled individualisable process program and is individually disconnectable without having to stop the device.

To this end the invention concerns a device for producing objects from biodegradable materials consisting of a storage tank with solid or liquid biodegradable material that is connected to a distribution line along which a series of baking forms with an individualisable form are filled, and whereby each baking form goes through an individualisable computer-controlled process program that determines the filling time, pressure and temperature, and afterwards automatically opens the baking form and removes the object formed, after which the next piece can be pressed or cast in the same baking form.

An advantage of such a device is that a number of types of objects can be produced simultaneously on one machine in an automated way, so that one operator can produce a number of baked forms simultaneously.

Preferably each baking form is individually disconnectable, whereby the operation of the entire device does not have to be stopped and the other baking forms continue producing while one baking form is replaced by a different one.

An advantage of this disconnectability is that a certain baking form can be used for a specific type of packaging, for example, that has to be produced in a smaller series after which this baking form can be replaced by a different one for a subsequent different series, and this without having to interrupt the production in the other baking forms, so that continuous production for 24 hours per day 7 days per week is possible.

Preferably each baking form is automatically controlled by a computer-controlled individual process program that ensures the opening of the supply pipe from the distribution line, the filling of the baking form, the control of the heating cycle in the baking form until the desired form has hardened, the opening of the baking form, the releasing of the object formed, the closing of the baking form and the automatic repetition of the steps for producing a next piece, and this until the desired series has been completed.

An advantage of such a computer-controlled process program is that the entire series of objects to be produced can be produced without manual assistance and without interruption due to the starting or stopping of other series in the other baking forms that are connected to the distribution line.

Preferably the baking form is opened by a computer-controlled electrically driven hydraulic ram that is connected via a pivot point to an eccentric point on a strip-shaped lever and which in a first phase opens the cover of the baking form that is in a closed state in a vertical position, by moving this cover horizontally over a small distance (10 mm), after which the cover is turned upwards over 90° using the strip-shaped lever that is fastened in a hinged way to the cover until the cover is in a horizontal position.

The object formed in the biodegradable material can now be automatically released and removed from the baking form.

A more preferred embodiment of the baking form, whereby the baking form is not closed by turning a cover down around a hinge located on the top of the baking form, but by the horizontal sideways movement of the male baking form section up to against the female baking form section.

The female baking form section is hereby in the main housing and the male baking form section is then pressed by means of a movable claw with four guide rods that is pushed against the male baking form section by a ram and locks the baking form.

An advantage of this embodiment is that the occurrence of leaks at the edges of the two baking form sections is prevented by the claw, and the lifetime of the baking form is improved.

Another advantage of this embodiment is that the movable male baking form section can be made lighter so that the weight of the baking form is limited, which not only fosters the lifetime of the baking form but also reduces the changeover time when changing baking forms.

The walls of the female baking form section are provided with internal cooling channels through which a coolant is driven. The side walls of the female baking form section are provided with two slots in which the male baking form section is slid and with which the correct positioning of the two baking form sections is ensured.

The female baking form section is also provided with a central supply opening for supplying the paste to be cast from a central supply line.

The male baking form section is provided with an electricity supply that makes contact with the female baking form section and with the heating elements that are present in the baking form.

The invention also concerns a special plug for this more preferred embodiment, consisting of a plastic socket provided with a screw thread, but whereby the cross-section of the screw thread does not present a sharp point but forms a trapezial cross-section, whose wider base is closer to the longitudinal axis of the plug.

Such a plug is provided for each of the four guide rods of the movable claw and this plug is screwed into the metal main housing of the female baking form section that is provided with a trapezial complementary thread so that the plug cannot be unscrewed.

The plug ensures that the metal guide rods of the movable claw cannot make direct contact with the metal main housing, which lengthens the lifetime of the guide rods.

The invention also concerns a method for producing objects from a biodegradable material that comprises the following steps:
The collection of biodegradable raw materials or biological waste products from agriculture;
the formation of a paste or liquid from the biodegradable raw materials by mixing in water and biodegradable additives;
the transport of the casting or pressing mixture to a storage tank of a device for the production of objects from biodegradable raw materials, as described in claim 1;
the connection of suitable baking forms to a distribution line of a device, as described in claim 1;
going through a production cycle as described in claim 3, whereby for each separate baking form a computer-controlled individual process program is gone through that is adapted to the object to be produced;
the collection and packaging of the objects produced per type and per size of series for sending to the consumers.

The biodegradable additives that are added to the degradable raw material are selected as a function of the properties to be obtained in the objects produced. For example, vegetable resins can be used as a binder or natural emulsifiers, while they remain fully biodegradable.

With the intention of better showing the characteristics of the invention, a preferred embodiment of a device for producing objects from a biodegradable material according to the invention is described hereinafter by way of an example, without any limiting nature, with reference to the accompanying drawings, wherein:

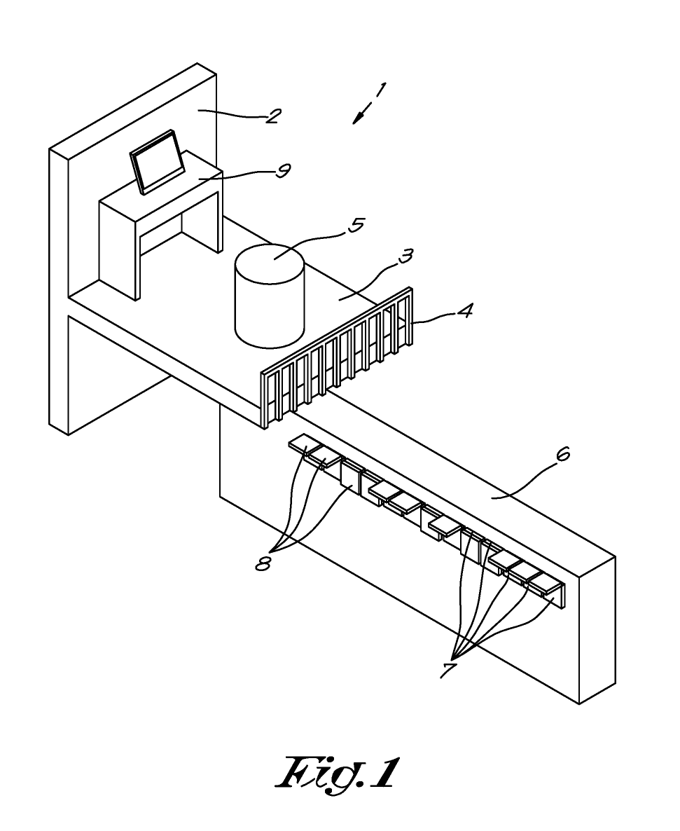

FIG. 1 schematically shows a perspective view of a device for manufacturing objects from biodegradable materials according to the invention;

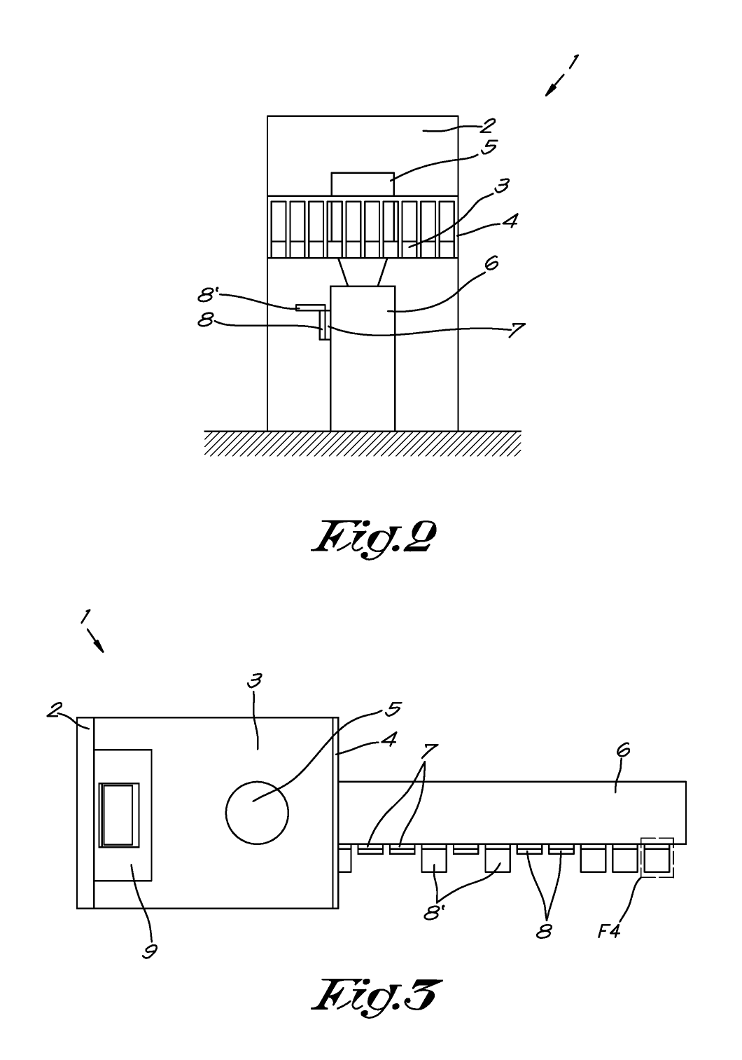

FIG. 2 shows a side view of FIG. 1;

FIG. 3 shows a top view of FIG. 1;

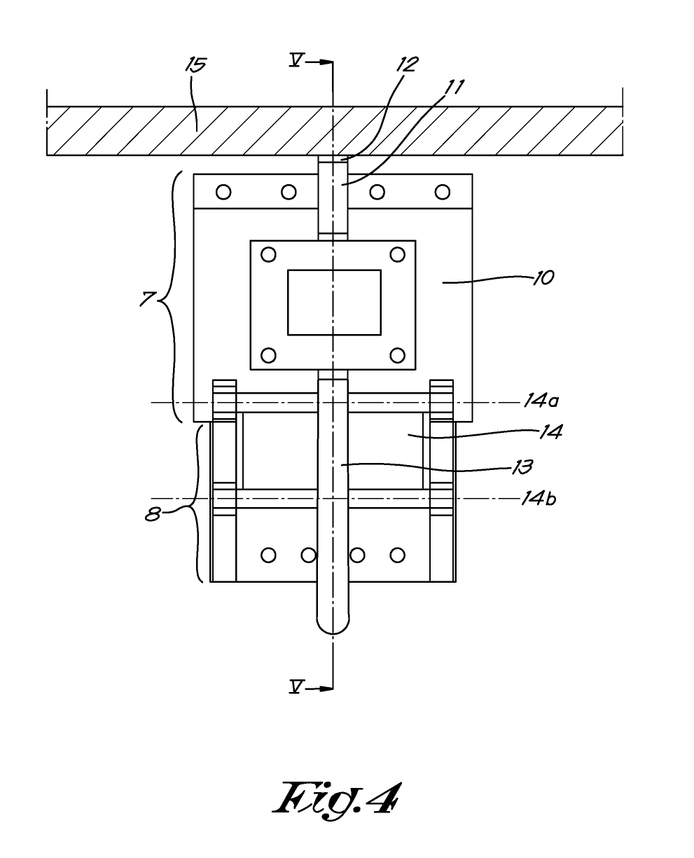

FIG. 4 shows the top view of a baking form indicated by F4 in FIG. 3 on a larger scale and in more detail;

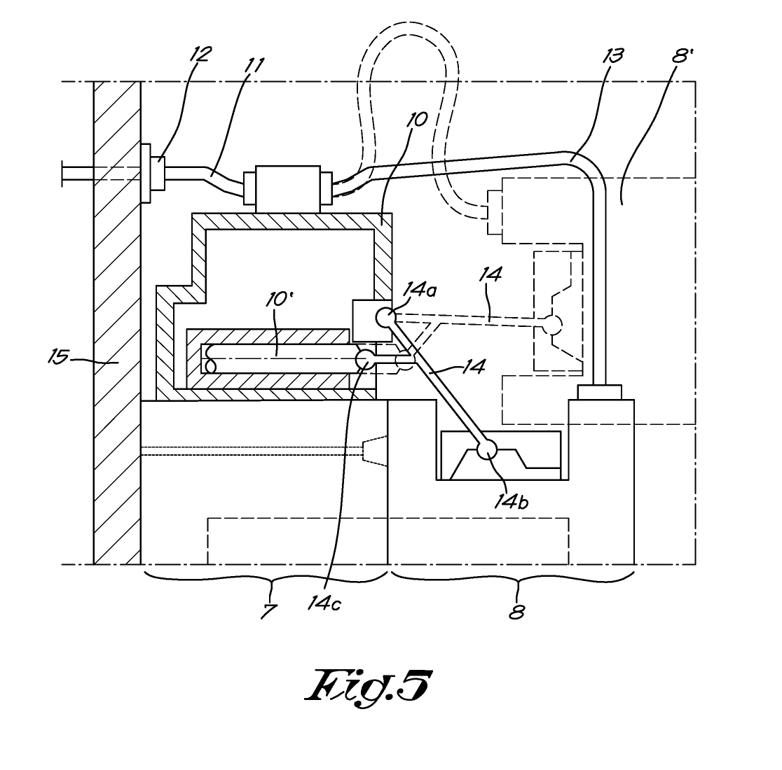

FIG. 5 shows a cross-section according to line V-V of FIG. 4;

FIG. 6 shows a perspective view of a few objects of biodegradable material produced with the device shown in FIG. 1;

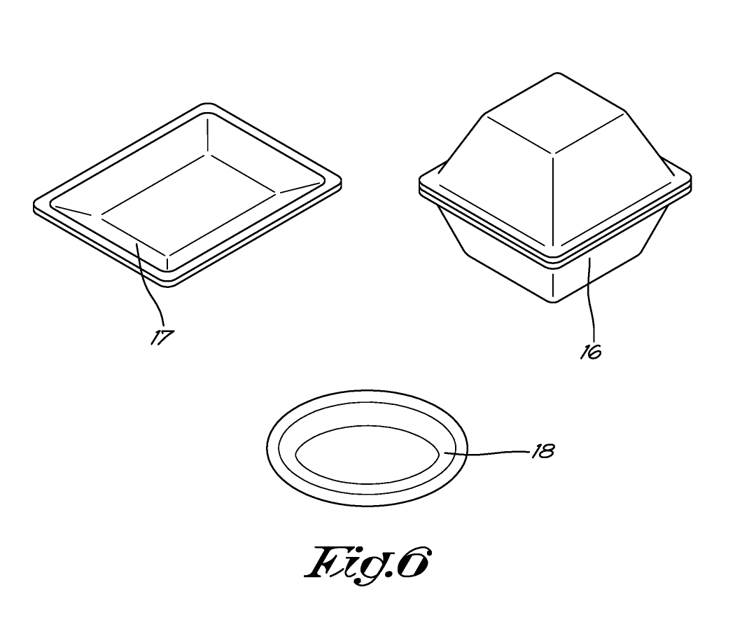

FIG. 7 shows a side view of a variant embodiment of one baking form;

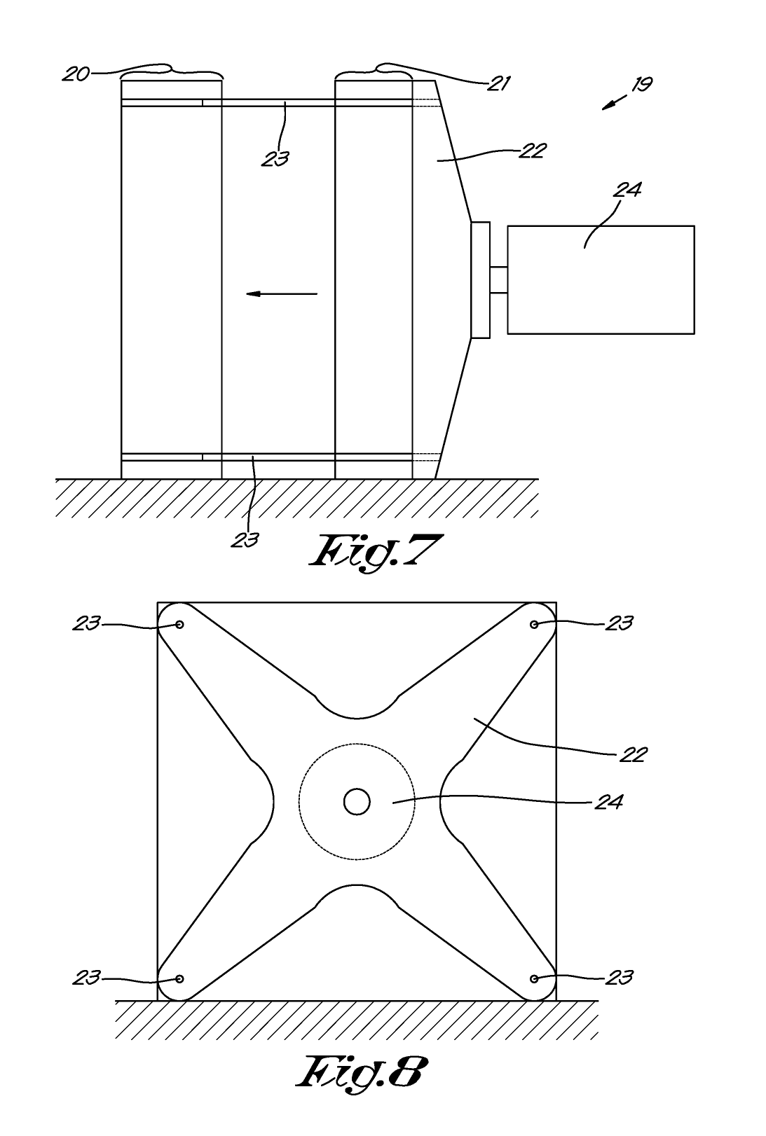

FIG. 8 shows a front view of FIG. 7;

FIG. 9 shows a side view of the double hermetic seal between the male and female baking form section;

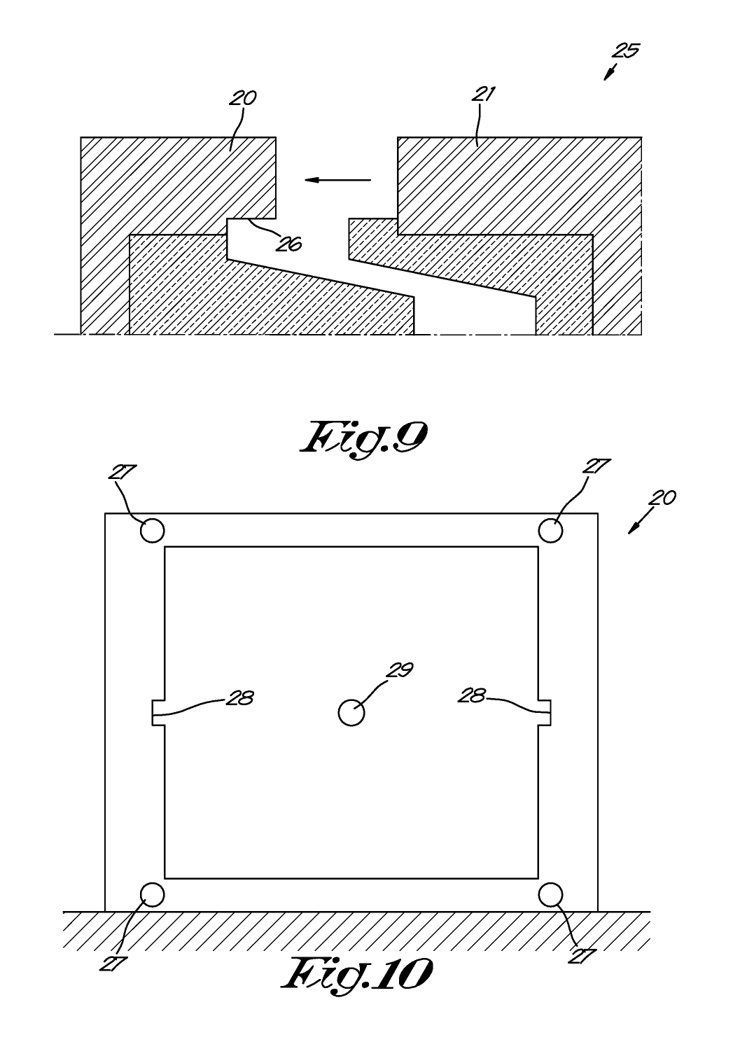

FIG. 10 shows a front view of the female baking form section of FIG. 7;

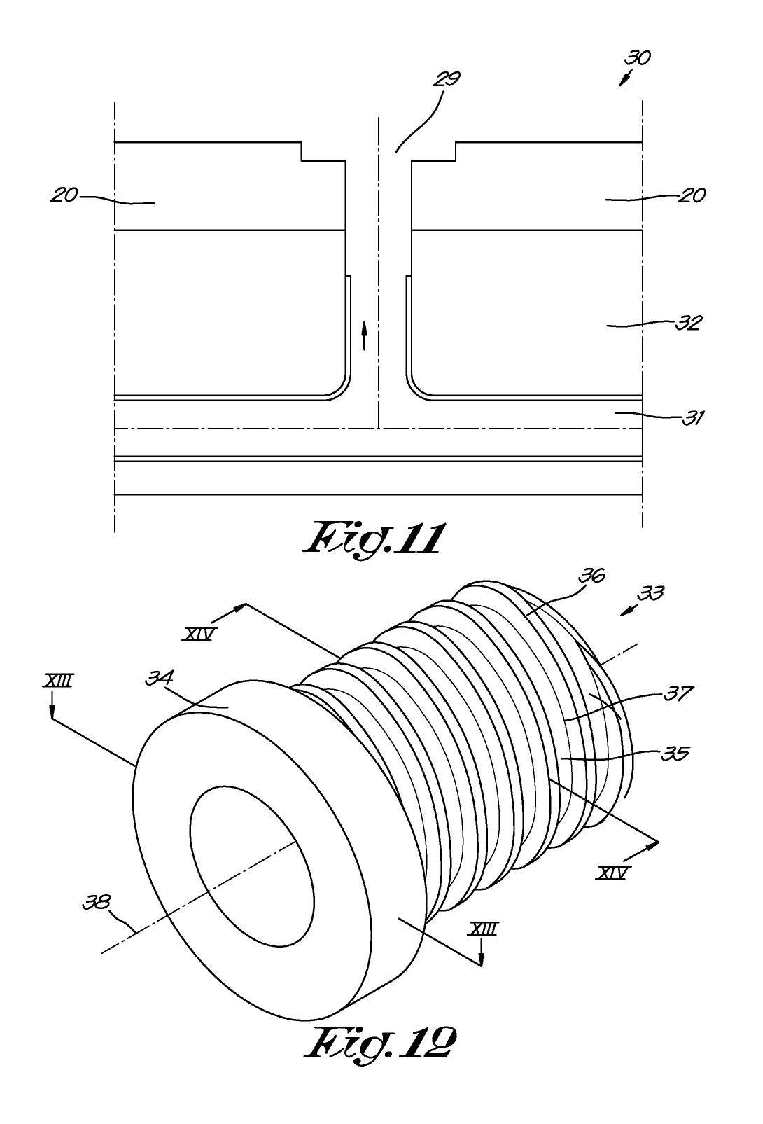

FIG. 11 shows a cross-section of the central supply channel for the casting paste in the female baking form section;

FIG. 12 schematically shows a perspective view of a plug with a trapezial cross-section according to the invention;

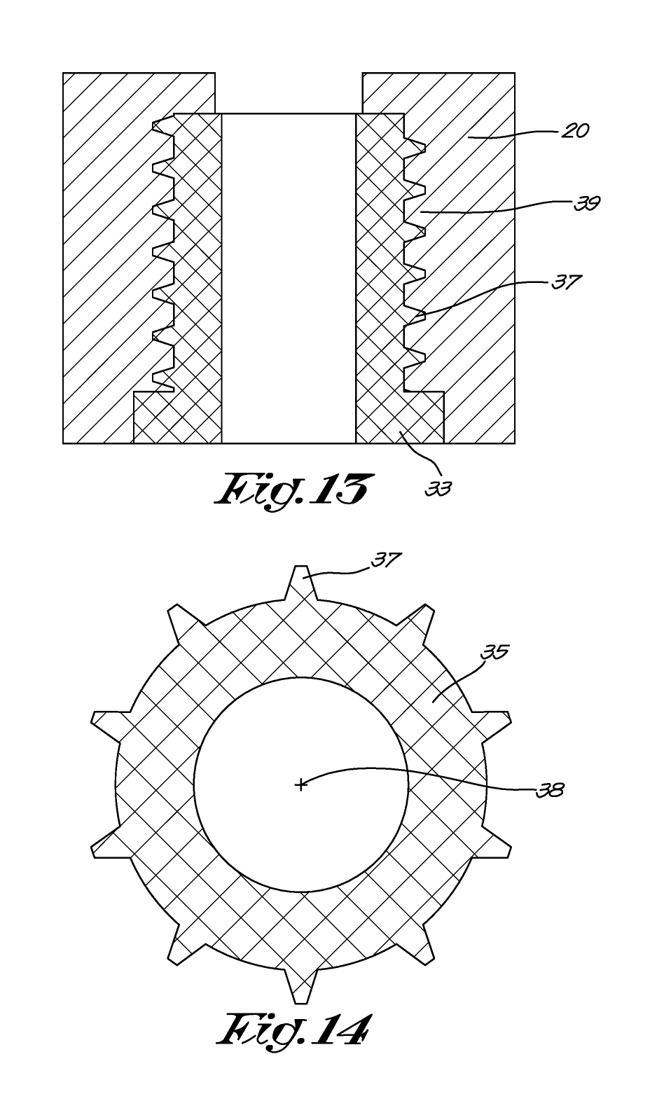

FIG. 13 shows a cross-section of FIG. 12 according to the line XIII-XIII;

FIG. 14 shows a cross-section of FIG. 12 according to the line XIV-XIV.

FIG. 1 shows a device for producing objects from biodegradable materials 1 according to the invention, consisting of a supporting framework 2 with a platform 3 provided with a guardrail 4, on which a storage tank 5 is placed for storing biodegradable material that is connected to a distribution line 6 that is disconnectably connected to each of the twelve baking forms 7 in this case, each of which is provided with a cover 8 and each is connected to a computer 9 with touchscreen from where the operation of the device can be controlled.

FIG. 2 shows a side view of FIG. 1 perpendicular to the longitudinal direction of the distribution line 6 to which the twelve baking forms 7 are connected. The cover 8′ of some baking forms is in a horizontal open position while other baking forms are closed whereby the cover 8 is in a vertical position.

FIG. 3 shows a top view of the device of FIG. 1, which shows which baking forms 7 are open whereby their cover 8′ is turned out to a horizontal position, while the others present their cover 8 in a closed vertical position.

FIG. 4 shows the top view of one baking form 7 in more detail, whose cover 8 is in a vertical closed position. Above the baking form 7 there is an electric housing 10 that is connected on the one hand to a computer 9 by a cable 11 with USB connection 12, and connected on the other hand to the cover 8 of the baking form 7 by a flexible electric cable 13. A strip-shaped lever 14 connects the horizontal axes 14a and 14b.

FIG. 5 shows a cross-section of FIG. 4 in which the mechanism can be seen by which the cover 8 of the baking form 7 can be placed in the open position 8′. There is a computer-controlled electrically driven hydraulic ram 10′ in the electric housing 10, that is connected via pivot point 14c to an eccentric point on a strip-shaped lever 14 that itself is mounted on bearings in a hinged way in the electric housing 10, on the one hand around axis 14a and on the other hand around axis 14b in the top of the cover 8 of the baking form in the closed vertical position or of the cover 8′ in the open horizontal position. The baking form 7 is fastened to an end plate 15 in which four openings are provided for supporting screws as well as four openings for the supply of biodegradable material to the baking form 7.

FIG. 6 shows a perspective view of a few objects that have been produced by means of the device presented in FIG. 1. A lunchbox 16 and a dish for noodles 17 are illustrated, as well as a dish for foodstuffs 18, that are all made from starch.

FIG. 7 shows a side view of a variant embodiment of baking form 19, consisting of a female baking form section 20 and a male baking form section 21, that can be slid against the female baking form section by means of a movable claw 22 with four guide rods 23 that is pressed against the male baking form section 21 by a ram 24.

FIG. 8 shows a front view of the movable claw 22 of FIG. 7, that is connected by four ends to the four guide rods 23 that fit in four cutaways provided to this end in the female baking form section 20.

FIG. 9 shows a side view of the sealing face 25 between the female baking form section 20 and the male baking form section 21 in more detail, whereby the double hermetic seal 26 between the male and female baking form sections can be seen.

FIG. 10 illustrates a front view of the female baking form section 20, in which the four cutaways 27 in the female baking form section 20 can be seen, in which the guide rods 23 are connected to the movable claw 22, and in which the two sidelong guide grooves 28 are shown, by which the internal baking form casing of the female baking form section 20 can be positioned. The central supply opening 29 for the paste to be cast is in the centre of the female baking form section 20.

FIG. 11 shows a cross-section of the supply 30 of the paste to be cast, that is distributed from a central supply pipe 31 to the baking form via a central supply opening 29 in the female baking form section 20 that is placed in the main housing 32.

FIG. 12 shows a plug 33 according to the invention, consisting of a flange-shaped section 34 and a socket-shaped section 35, that is provided with a screw thread 36 with a trapezial cross-section 37, whereby the widest base of the trapezial cross-section is oriented towards the central longitudinal axis 38 of the plug 33.

FIG. 13 shows a cross-section of FIG. 12 according to the line XIII-XIII, in which the trapezial cross-section 37 of the screw thread of the plug 33 can be seen, as well as the complementary thread 39 in the metal housing of the female baking form section 20, in which the plug 33 can be screwed.

FIG. 14 shows the trapezial cross-section 37 of the screw thread in the socket-shaped section 35 of the plug 33 in more detail.

The operation of the device 1 for producing objects from biodegradable materials according to the invention can be described as follows.

The storage tank 5 of the device 1 is filled with a paste of biodegradable material, such as vegetable starch, or with a liquid form of a biodegradable material such as lactic acid or citric acid mixed with glycerol, enzymes and other vegetable materials.

The biodegradable material is pressed or pumped in a baking form 7 in a continuous process, where the desired form is obtained and is then heated to the desired temperature whereby the object 17 is baked in a certain time such that it takes on its final form.

The continuous process is controlled by a control program in a computer 9 that can be operated by means of a touchscreen. The touchscreen shows all baking forms 7 that are connected to the storage tank 5 by one distribution line 6.

The computer 9 sends six basic instructions to each baking form 7 separately at the appropriate time:

1) the opening of the supply tap to the baking form;

2) the filling of the baking form 7 with the paste or solution;

3) the closing of the supply tap to the baking form;

4) the control of the baking time and the temperature in the baking form 7 according to the object being produced;

5) the opening of the baking form 7 by turning the cover 8 open and removing the produced object 17 from the baking form 7 in order to be received in a receptacle;

6) the closing of the baking form 7 by closing the cover 8 and restarting the production cycle for a next piece.

The opening of the baking form 7 by turning the cover 8 open is electronically controlled by the computer 9 through the movement of an electrically driven hydraulic ram 10′ that is connected via pivot point 14c to an eccentric point on a strip-shaped lever 14, which itself is mounted on bearings in a hinged way in the electric housing 10 around axis 14a on the one hand, and in the top of the cover 8 of the baking form around axis 14b on the other hand. In the first phase the ram 10′ pushes against the pivot point 14c whereby the cover 8 of the baking form 7 is moved over a small distance (10 mm) in a horizontal direction so that the cover 8 comes away from the baking form 7 in which the object is formed. In a second phase the ram 10′ pushes the strip-shaped lever 14 upwards in a hinged way over 90°, whereby the cover 8′ is now in a horizontal position, and the object produced is pulled from the baking form 7 by an eye-shaped hook (not shown) on each side of the baking form 7 after which the object 17 produced is guided to the desired conveyor belt.

The closing of the baking form 7 is controlled by the computer 9 by going through the above movements in the reverse order, after which the cycle is repeated for a next piece until the desired number of objects has been produced. The duration of the production cycle of one object can vary from 15 seconds to 45 seconds depending on its dimensions. The baking forms 7 and covers 8 are made of cast aluminium and have an anodised non-stick layer on the contact surface with the object to be produced. The baking form 7 consists of a female section that is fastened immovably to the end plate 15, and a male section that forms part of the movable cover 8. The weight of a baking form varies from 10 to 16 kg depending on the object to be formed. Both the female section and the male section of the baking form 7 are provided with an electric heating coil that is controlled by the process program in the computer 9 and whereby each baking form goes through a programmed temperature cycle separately that is individually adjustable per baking form 7.

The baking form can also have an alternative embodiment 19, whereby the hinged cover with the male baking form section is replaced by the horizontal sideways movement of the male baking form section 21, up to against the female baking form section 20.

The female baking form section 20 is hereby in the main housing and the male baking form section 21 is pressed by means of a movable claw 22 with four guide rods 23 that is pushed against the male baking form section 21 by a ram 24 and locks the baking form 19, whereby the contact surface between the two baking form sections is sealed in a leakproof way by means of a double hermetic seal 26.

In this embodiment the movable male baking form section 21 is made lighter, so that the weight of the baking form 19 is reduced from 390 kg to 18 kg, which not only favours the lifetime of the baking form 19, but also reduces the changeover time when changing baking forms.

In this embodiment, upon closing the baking form the movable male baking form section 21 makes contact with the electricity supply on the main housing, such that the heating elements in the male baking form section 21 are supplied with electricity during the baking phase.

The metal housing of the female baking form section is provided with a complementary thread with a trapezial cross-section, in which a plastic plug can be screwed for each guide rod, that is provided with a screw thread with a trapezial cross-section in order to prevent contact between the metal guide rods and the metal housing.

According to the invention such a plug cannot be unscrewed and can provide long-term protection for the guide rods by being made of nylon for example.

The computer 9 also controls the press or the pump that supplies the distribution line 6. The operator can increase the pressure in the distribution line according to the number of baking forms 7 that are in use. The speed of the supply flow can be controlled according to the weight of the objects to be produced per unit time. The control program can simultaneously control up to 30 baking forms in one distribution line of a device. A number of devices can be deployed parallel to one another such that a high capacity production line can be formed.

It goes without saying that the distribution line 6 of a device can not only be horizontal, but also vertical or in other orientations depending on the circumstances.

It has also turned out to be advantageous to incorporate the computer in the control unit and to locate it next to the production line, so that the operator himself can physically oversee the production line at the same time.

The present invention is by no means limited to the embodiment described as an example and shown in the drawings, but a device for producing objects from biodegradable materials according to the invention can be realised in all kinds of forms and dimensions, without departing from the scope of the invention.

1-9. (canceled)

10. Device (1) for producing simultaneously a plurality of different objects from biodegradable materials comprising a storage tank (5) with solid or liquid biodegradable material that is connected to a distribution line (6) along which a series of baking forms (7) with an individualisable form, wherein each baking form (7) is individually and automatically controlled by a computer, and each baking form (7) is individually disconnectable, whereby the operation of the entire device (1) does not have to be stopped and the other baking forms (7) continue to produce, while one baking form is replaced by a different one.

11. Production process using the device described in claim 10, wherein the production process is automatically controlled by a computer (9) for each baking form (7) using an individual process program, that ensures
the opening of the supply pipe of the distribution line (6);
the filling of the baking form (7);
the control of a heating cycle in the baking form until the desired form has hardened;
the opening of the baking form;
the release of the object (17) formed;
the closing of the baking form (7) and;
the automatic repetition of these steps for producing a next piece, and this until the desired series has been completed.

12. Device according to claim 10, wherein
the baking form (7) is opened by means of a computer-controlled electrically driven hydraulic ram (10′) that is connected via a pivot point (14c) to an eccentric point on a strip-shaped lever (14), which in a first phase opens the cover (8) of the baking form that is in a vertical position, by moving this cover horizontally over a small distance (10 mm) after which the cover is turned upwards over 90° using the strip-shaped lever (14), which on the one hand is mounted on bearings around a horizontal axis (14b) in the cover (8) and on the other hand is mounted on bearings around a horizontal axis (14a) in the electric housing (10), until the cover (8′) is in a horizontal position.

13. Method for producing objects from a biodegradable material, which comprises the following steps:
the collection of biodegradable raw materials or biological waste products from agriculture;
the formation of a paste or liquid from the biodegradable raw material by mixing in water and biodegradable additives;
the transport of the casting or pressing mixture to a storage tank of a device for the production of objects from biodegradable raw materials;
the connection of suitable baking forms to a distribution line of a device;
going through a production process cycle as described in claim 11, whereby for each separate baking form a computer-controlled individual process program is gone through that is adapted to the object to be produced;
the collection and packaging of the objects produced per type and per size of series for sending to the consumers.

14. Device according to claim 10, wherein the baking form (19) is opened by means of a computer-controlled ram (24) that is connected to a movable claw (22) with four guide rods (23) that is pressed against the male baking form section (21) and locks the baking form (19), whereby the contact surface between the two baking form sections is sealed in a leakproof way by means of a double hermetic seal (26).

15. Device according to claim 14, wherein the supply (29) of the paste to be cast is distributed to the baking form from a central supply pipe (30) via a central supply opening (31) in the female baking form section (20) of the baking form (19).

16. Device according to claim 14, wherein when closing the baking form (19) the male baking form section (21) makes contact with the electricity supply on the main housing (32), such that the heating elements in the male baking form section (21) are supplied with electricity during the baking phase.

17. Device according to claim 14, wherein
the metal housing of the female baking form section is provided with a complementary thread with a trapezial cross-section, in which a plastic plug can be screwed for each guide rod, that is provided with a screw thread with a trapezial cross-section, to prevent contact between the metal guide rods and the metal housing.
